(19) United States
(12) Patent Application Publication
(10) Pub. No.: US 2011/0251523 A1
(43) Pub. Date: Oct. 13, 2011

(54) SKIN CARE APPARATUS AND METHOD OF SKIN CARE USING THE SAME

(75) Inventor: Young Ho KIM, Seoul (KR)

(73) Assignee: Elcos America, Inc.

(21) Appl. No.: 12/756,292

(22) Filed: Apr. 8, 2010

(51) Int. Cl. A61N 5/06; A61N 7/00

(52) U.S. Cl. 601/2; 607/88

(57) ABSTRACT

The present invention's effective treatment is based on ultrasound, ion delivery and far infrared rays, the beneficial energy of the ions attack the bacteria of the skin and strengthens the capabilities of cellular resistance to increase skin elasticity, reduces visibility of pores and smoothes the skin, Far Infrared Rays improves the circulation of blood vessels and speeds skin metabolism, the ultrasound's micro massage feature strengthens the elasticity of the skin, rendering skin younger and more luminous.

### BACKGROUND OF THE INVENTION

1. Field of the Invention

The present invention relates to a skin care device and a method of skin care suing the skin care device.

2. Description of the Related Art

The skin is vulnerable to skin problems. Even those individuals who have not suffered from skin problems in their younger years will eventually begin to suffer skin aging and skin aging symptoms such as fine lines, wrinkles, sagging facial skin, age or liver spots, and possibly skin cancer. There have been various ways to remedy those problems and restore the healthy skin Particularly, the skin treatments at a doctor's office or at a spa rendering them are very expensive. A single device that eliminates expensive treatments at a doctor's office or at a spa is needed, and that is able to stabilize skins biological structure and increase its elasticity to give it a luminous tone is needed.

### SUMMARY

An embodiment of the present invention provides a skin care apparatus which includes an infrared ray generator generating an infrared ray generator, an ultrasound generator generating ultrasound, an ion delivery device generating and delivering an ion, a first output part through which the generated ultrasound and the generated ion are outputted, a second output part through which the generated infrared ray is outputted, and a controller controlling the infrared ray generator, the ultrasound generator, and the ion delivery device in accordance with a preprogrammed mode or in accordance with the user's selection, the controller capable of operating concurrently at least two of the infrared ray generator, the ultrasound generator, and the ion delivery device.

In an embodiment of the present invention, the infrared ray generator generates far-infrared ray with the wavelength of 8-14 μm.

In an embodiment of the present invention, the ultrasound generator generates about 5 million vibrations per second.

In an embodiment of the present invention, the ion delivery device generates galvanic ions.

In an embodiment of the present invention, the controller is capable of operating the ultrasound generator and the infrared ray generator concurrently and operating the ion delivery device and the ultrasound generator concurrently.

In an embodiment of the present invention, the preprogrammed mode includes a spot care mode and a wrinkle care mode.

In an embodiment of the present invention, there are a wrinkle care mode switch for selecting the wrinkle care mode and a spot care mode switch for selecting the spot care mode.

In an embodiment of the present invention, the skin care apparatus further includes a tri-function indicator to indicate a status of the infrared ray generator, a status of the ultrasound generator, and a status of the ion delivery device.

Another embodiment of the present invention provides a method of skin care using the skin care apparatus. The method includes: removing and arranging keratin by operating the ultrasound generator and the infrared ray generator concurrently; performing at least one of whitening a skin of a user by operating the ion delivery device and the ultrasound generator concurrently and caring wrinkles by vibration and ultrasonic wave generated from the ultrasound generator; and supplemental-treating the skin by vibration after whitening and by vibration and ultrasonic wave generated by the ultrasound generator after caring the wrinkles.

### BRIEF DESCRIPTION OF THE DRAWINGS

The above and other features and advantages of the present invention will become more apparent by describing in detail exemplary embodiments thereof with reference to the attached drawings in which:

FIG. 1 is a perspective view of a skin care apparatus according to an embodiment of the present invention;

FIGS. 2A and 2B illustrate the effects of the positive ion when using a skin care apparatus according to an embodiment of the present invention;

FIGS. 3A and 3B illustrate the effects of the negative ion when using a skin care apparatus according to an embodiment of the present invention;

FIG. 4 is a graph illustrating electroconductivity via novameter according to Examples of the present invention; and

FIGS. 5A and 5B show the test product questionnaire summary according to Examples of the present invention.

### DETAILED DESCRIPTION

According to an embodiment of the invention, a skin care apparatus includes an infrared or far-infrared ray generator, an ultrasound generator, and an ion delivery device.

The infrared or far-infrared ray generator generates an infrared or far-infrared ray. The ultrasound generator generates ultrasound. The ion delivery device generates an ion. According to an embodiment of the present invention, the generated ultrasound and the generated ion are outputted through a first output part, and the generated infrared or far-infrared ray is outputted through a second output part.

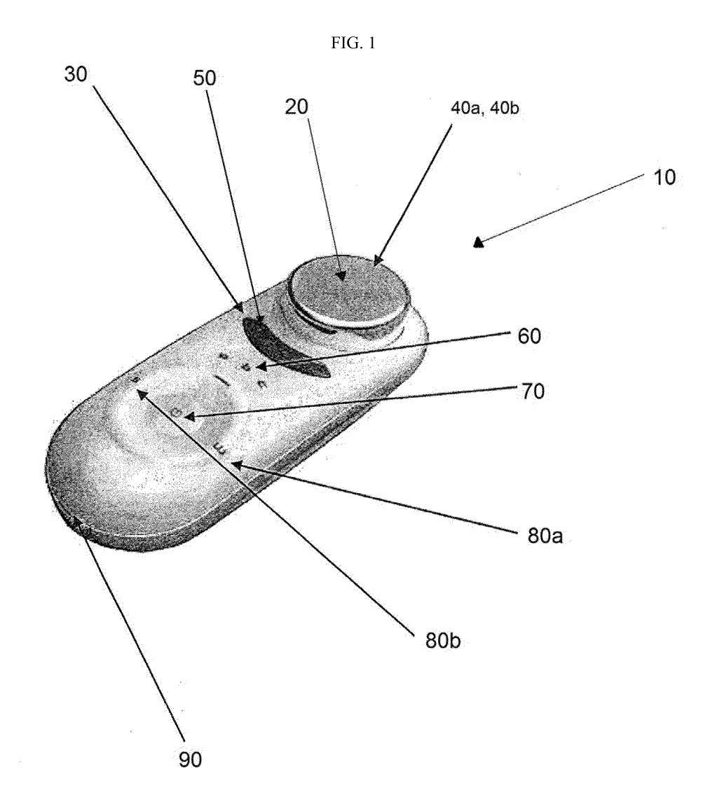

In an embodiment according to the present invention illustrated in FIG. 1, the skin care apparatus 10 includes the first output part 20 through which the ultrasound and the ion is outputted, and the second output part 30 through which the infrared ray is outputted.

The ultrasound generator 40a generates ultrasonic wave. Ultrasonic wave generates preferably about 5 million vibrations per second (5 MHz) which makes skin cell's natural healing properties activate and helps your skin fully recover. Micro massage strengthens the elasticity of the skin, rendering it more youthful and luminous. This vibration pushes nutrients into the deepest layer of skin.

In addition, the heat generated by the intensive ultrasonic waves raises skin temperature by 0.5 to I degrees Celsius, accelerates blood and lymph streams, and promotes absorption of nourishment.

The ultrasonic wave opens pores and loosens years of makeup buildup, blackheads, whiteheads, dirt and oil leaving skin clean and youthful. Ultrasound promotes production of collagen and elastin, which will keep your skin moist and elastic.

Our skin renews itself about every 28 days, ultrasonic waves help in removing dead skin cells. Ultrasonic wave restores vitality of fibrous tissues and makes wrinkles smoothed, sloping skin gets back elastic properties.

The infrared ray generator 50 generates infrared ray, more preferably, far-infrared ray (FIR) with the wavelength of 8-14 μm. The effects of the far infrared rays include a warming effect, metabolism increase, an anti-aging factor, an overall improvement and the production of healthy hormones.

As for the warming effect, far infrared rays warm the target area, unclogging pores & expanding capillaries, thereby promoting increased blood flow. The increase in blood flow helps to eliminate toxins, poisons, acids, bacteria, fungi, parasites and subcutaneous fats associated with aging and fatigue

As for the metabolism increase, the Far Infrared Rays also stimulates enzyme activity and metabolism in the target area, and generates a special heat that has been proven to breakdown cellulite, fat, trapped water and Wastes.

As for the anti-aging factor, far infrared light stimulates the production of cell tissue and rapidly promotes the regeneration of skin and blood tissue in areas exposed to its healing rays. It speeds cellular metabolic rates by stimulating the activity of mitochondria, and triggering enzyme activity.

As for the overall improvement, regular exposure to the FIR is similar to physical exercise in its effects on the body. Improving the balance of blood pressure, blood sugar, and an increase in oxygen and nutrients in your cells, resulting in increased muscle strength vitality and mental stability.

As for the production of healthy hormones, FIR is converted by the body into electrochemical impulses and sent to the pineal pituitary glands, which in turn produces healthy hormones that are sent through the central nervous system to all cells in the body, thus this special light is essential to functioning of our entire endocrine system.

The ion delivery device 40b generates ion, more preferably galvanic ion.

The ion delivery device 40b uses a potential difference between two metal characteristic materials. When the electrolyte solution is come into between two metal characteristic materials, the positive ion wants to move forward to the negative ion, and the negative ion wants to move forward to the positive ion. The effects of the ion delivery are explained hereinbelow.

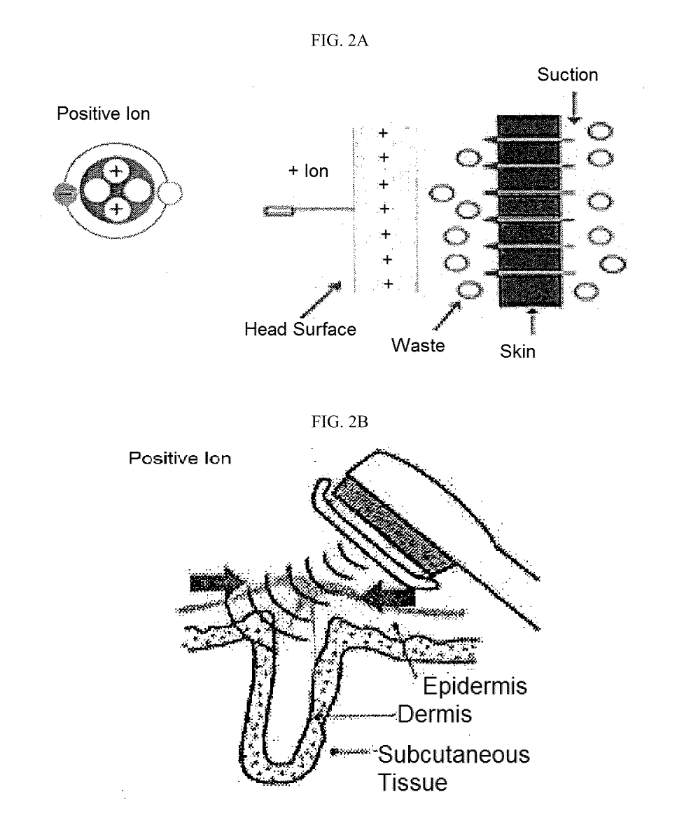

The beneficial energy of the ions attacks bacteria and strengthens the capability of the skins cellular production. The ion delivery device also has the cleansing effect. Positive Ions cling onto makeup, dirt, oils and other foreign matters, the electric properties extracts the impurities from deep within the skin. The positive ions close pores and help reduce oil production. (See FIGS. 2A and 2B.)

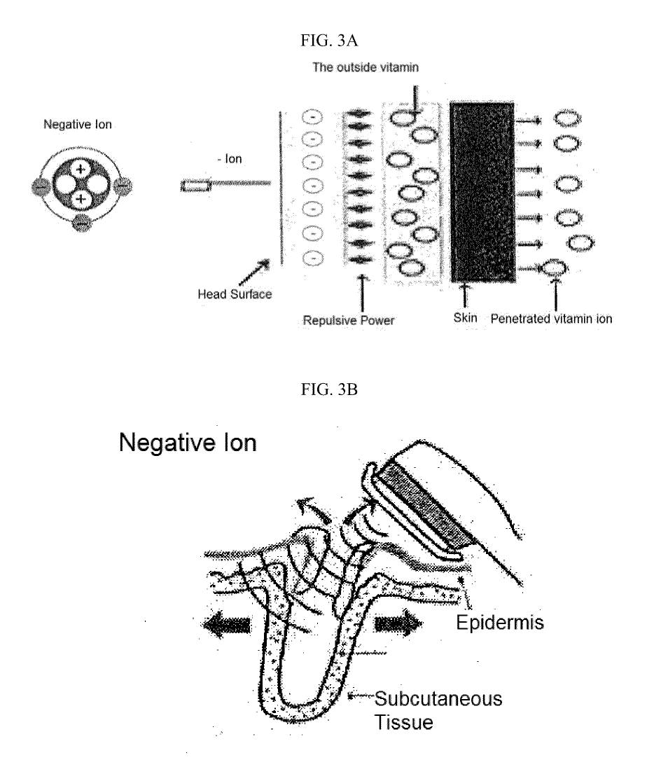

Negative ions attach to the cream/serum and push the beneficial nutrients deep into the layers of the skin. The negative ions help open the pores and improve elasticity. (See FIGS. 3A and 3B.)

The beneficial nutrients that are deposited deep into the skin layers create fully hydrated skin cells.

The function of the device is exemplified in TABLE 1.

TABLE 1

| Process | Function | Mechanism | Details |
|---|---|---|---|
| Step 1 | Removal and | Ultrasonic + | Ultrasonic can open the |
| (Prepa- | Arrangement | Far infrared | pores of skin lightly |
| ration) | of Keratin | ray (concurred) | Boosting efficacy of |
| | | | enzyme |
| Step 2 | Whitening | Ion + | Boosting ingredients of |
| Activity | (Clear) | Ultrasonic | efficacy by enzyme and the |
| | | (concurred) | process that ultrasonic |
| | | | opens the pore are crossed |
| | | | over then efficacy ingre- |
| | | | dient is absorbed. |
| | Wrinkle | Vibration + | Ultrasonic: Protect aging of |
| | (Elastic) | Ultrasonic | skin/removal wrinkles |
| | | (Concurred) | Vibration: Elastic of skin |
| | | | and massage/Relax of mind |
| Step 3 | Supple- | Vibration after | On the base step, ion is |
| Finishing | mental | ion (Whitening)/ | penetrated into skin, the |
| | Barrier | Vibration + | ion is disappeared and the |
| | | Ultrasonic | vibration is exercise. |
| | | (Removal | |
| | | wrinkle) | |

The skin care apparatus according to an embodiment of the present invention may have a power switch 70, a mode switch such as a wrinkle care mode switch 80a and a spot care mode switch 80b, and a battery recharger area with a closure 90. The skin care apparatus may have additionally a tri-function indicator 60.

Briefly speaking, the galvanic ion helps absorption of formula and removes waste inside pores of skin. The ultrasound opens pres of skin, then pushes out the waste from the pores by vibrating, and it has efficacy of lipolysis by heating and vibrating. The far infrared ray can help the circulation of the blood so that it makes a cell by expanding of capillary. If we use these function in an optimized way, the efficacy will be substantially increased.

According to an embodiment of the present invention, the device may be used as follows. The user may apply serum/cream to cheek, and the user may be select one of the preprogrammed modes such as a spot care mode and a wrinkle care mode. The user may repeat these steps for the other parts of a face.

Examples of the method of using an embodiment of the present invention will now be described. However, the present invention is not limited to these examples.

### EXAMPLES

AMA Laboratories, Inc (New York, N.Y.) conducted the controlled usage study to evaluate tolerance and efficacy of the skin care apparatus according to an embodiment of the present invention.

The panel was convened to evaluate the efficacy of topically applied products intended to reduce fine lines and wrinkles, improve skin's elasticity and increase skin moisturization when tested over a 28 day consumer in-use regimen. Efficacy was evaluated for each subject using panelist self-assessment questionnaire and instrumentally via Nova Dermal Phase Meter.

1.0 Test Material:

1.1 Test Sample Description:

Eleven test samples were received from APRIORI Beauty, LLC. (Laguna Hills, Calif.)

| AMA Lab Nos.: | Client Nos.: |
|---|---|
| L-5293 | Silver Syringe, Precision Skin Rejuvenator |
| L-5294 | Cellular Age Advantage, Precision Skin |
| L-5295 | Rejuvenator, Lot #: C056 |
| L-5295 | Creme Cleanse 1, Lot #: C135 |
| L-5296 | Activating Tonic 2, Lot #: Cl08 |
| L-5297 | Firming Complex 3, Lot #: C130 |
| L-5298 | Eye + Lip Age Eraser 4, Lot #: C060 |
| L-5399 | Day Defense Plus 5, Lot #: C080 |
| L-5300 | Night Repair 3 Deep, 5, Lot #: Cl14 |
| L-5302 | Derma Firm Illuminator (Beauty Gadget) |
| L-5303 | Charger |
| L-5304 | Converter, 15 A, 250 V |

1.2 Handling:

Upon arrival at AMA Laboratories, Inc., the test materials were assigned a unique laboratory code number and entered into a daily log identifying the lot number, sample description, sponsor, date received and test requested. Samples are retained for a period of three months beyond submission of final report unless otherwise specified by the sponsor or if sample is known to be in support of governmental applications, in which case retained samples are kept two years beyond final report submission. Sample disposition is conducted in compliance with appropriate federal, state and local ordinances.

1.3 Test Material Evaluation Prerequisite:

Prior to sample submission to AMA the following tests were conducted with no adverse results:
USP or CTFA Preservative Efficacy Test or equivalent
90 Day Accelerated Stability and Container Compatibility Study
Fifty (50) person Repeat Insult Patch Test (RIPT) or equivalent

2.0 Institutional Review Board:

Reference: CFR Title 21 Part 56, Subparts A, B, C, and D. The IRB of AMA Laboratories, Inc., consists of 5 or more individuals, chosen from within the company for technical expertise and from the local community for lay interaction.

3.0 Panel Selection:

3.1 Standards for Inclusion in a Study:
1. Individuals who will complete a preliminary medical history mandated by AMA Laboratories, Inc.
2. Individuals who will read, understand and sign an informed consent document as required by Reference 21 CFR Ch. 1 Part 50, Subpart B.
3. Individuals in good health and free of any dermatological or systemic disorder, which would interfere with the results, at the discretion of the Investigator.
4. Individuals, who will be able to cooperate with the Investigator and research staff, be willing to have the test materials applied according the protocol and complete the full course of study.
5. Individuals with no known abnormal response to cosmetic products and who are willing to cooperate with the study requirements.
6. Individuals who have abstained from using any anti-aging and moisturizing treatment products for a period of 72 hours prior to study commencement and during the test period.

3.2 Standards for Exclusion from a Study:
1. Individuals who are under doctor's care.
2. Individuals taking medication which in the opinion of the Investigator would mask or interfere with the results.
3. Individuals with chronic skin allergies or skin conditions such as severe dermatitis, psoriasis, and/or eczema.
4. Individuals with uncontrolled diseases such as diabetes, hypertension, hyperthyroidism, or hypothyroidism.
5. Individuals with irritation or sensitivity to cosmetic products, soaps, shampoos or personal hygiene cleansing products.
6; Female volunteers who indicate that they are pregnant or lactating.
7. Individuals with blemishes, nevi, sunburn, suntan, scars, moles, active dermal lesions or uneven pigmentation in the test sites.
8. Individuals participating in any clinical research study at another facility or with a doctor's office at the commencement and duration of the study.

3.3 Recruitment:

Panel selection is accomplished by advertisements in local periodicals, community bulletin boards, phone solicitation, electronic media or any combination thereof.

4.0 Population Demographics:

| Number of subjects enrolled | | 5 |
|---|---|---|
| Number of subjects completing study | | 5 |
| Age Range | | 41-46 |
| Sex | Female | 5 |
| Race | Caucasian | 5 |

5.0 Procedure:

Five female subjects were inducted into this study. The demographic data is shown in Section 5.0. All subjects completed a screening form, medical history form, and informed consent document prior to commencement. In order to pre-condition the test sites and keep all topical treatments consistent during the study, the panelists were required to abstain from using any antiaging and moisturizing products for a period of 72 hours prior to study commencement and to use only the assigned test materials throughout the study period.

All subjects were required to use the test materials as part of their normal daily routine according to the sponsor supplied use instructions (see attached addendum).

This application regimen was conducted for a 28 day period. Participants were provided with a daily log and were instructed to record the time of each application together with any subjective comments regarding product usage.

On the day of the study panelists reported with their face and neck devoid of topical treatments. Upon arrival, panelists were allowed to equilibrate to the ambient environment for 30 minutes prior to measurement.

Biophysical measurements were collected prior to the initial application and again after 10 minutes, 7, 14 and 28 and days of treatment. In addition participants were asked to fill out a self-assessment questionnaire at each evaluation point.

The following distinct noninvasive method was employed to establish evaluation parameters:

### Electroconductivity—Novameter

A Nova Dermal Phase Meter, Model DPM 9003 (Nova, Technology Corp., Gloucester, Mass.) was used to obtain measurements of skin surface impedance to determine electroconductivity of the treatment sites. This meter provides a relative measure of the retained water content of the skin as a function of the skin's dielectric value. Skin impedance was recorded automatically when equilibrium was achieved.

Clients are notified immediately in the case of an adverse reaction and a determination is made as to treatment program if necessary.

5.0 References:
Leveque, J. L., de Rigal, J.: Impedance Methods for Studying Skin Moisturization, J. Soc. Cosmet. Chem., 34: 419-428, 1983, which is incorporated herein by reference.

6.0 Statistical Source Data:

The source data is Novameter readings taken prior to application and again after 10 minutes, 7, 14 and 28 days of use. In addition a self-assessment questionnaire was administered. The data used in the statistical analysis reflects changes from baseline.

7.0 Results:

Electroconductivity via Novameter (L5293-6304)

| AMA lab Nos.: | Client Nos.: |
|---|---|
| L-5293 | Silver Syringe, Precision Skin Rejuvenator |
| L-5294 | Cellular Age Advantage, Precision Skin |
| | Rejuvenator, Lot #: C056 |
| L-5295 | Creme Cleanse 1, Lot #: CBS |
| L-5296 | Activating Tonic 2, Lot #: CI08 |
| L-5297 | Firming Complex 3, Lot #: C130 |
| L-5298 | Eye + Lip Age Eraser 4, Lot #: C060 |
| L-5299 | Day Defense Plus 5, Lot #: C080 |
| L-5300 | Night Repair 3 Deep, 5, Lot #: C114 |
| L-5302 | Derma Firm Illuminator (Beauty Gadget) |
| L-5303 | Charger |
| L-5304 | Converter, 15 A, 250 V |

| | Baseline | 10 Minutes | Day 7 | Day 14 | Day 28 |
|---|---|---|---|---|---|
| 58 7412 | 275.67 | 321.67 | 367.67 | 403.33 | 462.33 |
| 66 4354 | 139.00 | 233.67 | 259.67 | 229.34 | 328.00 |
| 46 4934 | 165.34 | 346.34 | 321.67 | 337.67 | 382.33 |
| 62 2993 | 198.34 | 388.00 | 229.00 | 347.67 | 395.50 |
| 66 9598 | 340.67 | 380.67 | 230.67 | 310.33 | 348.67 |
| Mean | 223.80 | 334.07 | 281.74 | 325.67 | 383.37 |

| | | | | |
|---|---|---|---|---|
| % Difference | 49.27% | 28.59% | 45.51% | 71.30% |
| % Max Improvement | 109.47% | 94.55% | 104.23% | 131.24% |
| P | 0.026* | 0.283 | 0.046* | 0.014* |
| T | 3.434* | 1.239 | 2.853* | 4.170* |

*Statistically Significant

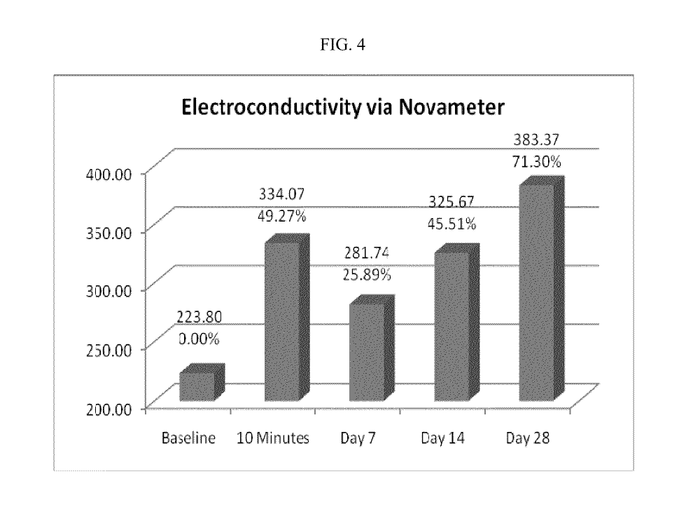

Electroconductivity via Novameter is also shown in FIG. 4.

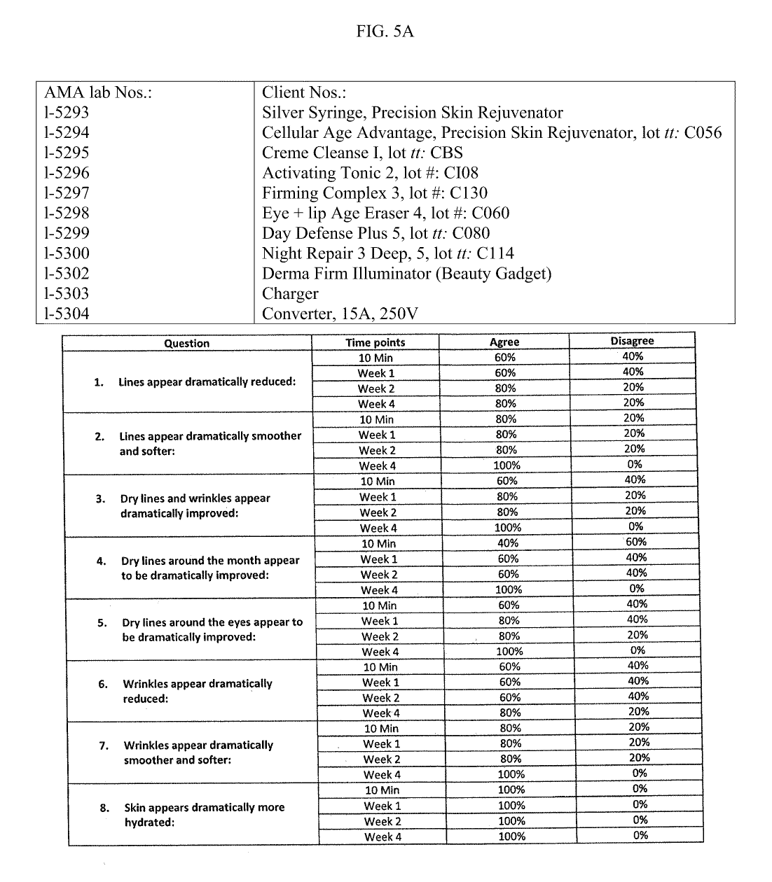

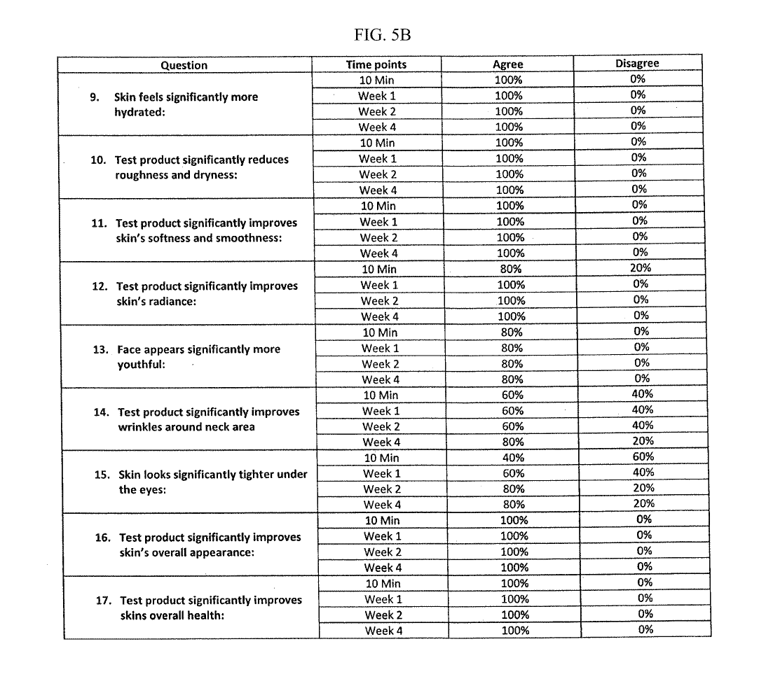

Test Product Questionnaire Summary is shown in FIGS. 5A and 5B.

8.0 Observations:

No adverse effects or unexpected reactions of any kind were observed on any of the subjects.

9.0 Conclusions:

Within the limits imposed by the conduct and population size of the study described herein, the following conclusions may be drawn.

Novameter readings demonstrated that the following test materials:

| AMA Lab Nos.: | Client Nos.: |
|---|---|
| L-5294 | Cellular Age Advantage, Precision Skin |
| | Rejuvenator, Lot #: C056 |
| L-5295 | Creme Cleanse 1, Lot #: CBS |
| L-5296 | Activating Tonic 2, Lot #: Cl08 |
| L-5297 | Firming Complex 3, Lot #: C130 |
| L-5298 | Eye + Lip Age Eraser 4, Lot #: C060 |
| L-5299 | Day Defense Plus 5, Lot #: C080 |
| L-5300 | Night Repair 3 Deep, S, Lot #: C114 |

when supported with following devices:

| AMA Lab Nos.: | Client Nos.: |
|---|---|
| L-5293 | Silver Syringe, Precision Skin Rejuvenator |
| L-5302 | Derma Firm Illuminator (Beauty Gadget) |
| L-5303 | Charger |
| L-5304 | Converter, 15 A, 250 V |

increased the skin moisture content 71.3% after 28 days with maximum of 131.24% observed. Below is the summary of the mean differences observed and statistical significance at each evaluation.

Subjective questionnaire responses corroborated instrumental data. Throughout the test period, the majority of test panelists perceived the test product to be an effective anti-aging and moisturizing product.

As shown above, the present invention is the one of the most technologically advanced home-use instrument and skin care treatment designed for the reduction and prevention of facial wrinkles and the overall improvement of skins vitality. The present invention helps reduce the depth of wrinkles, improves the texture and brightness of skin, and helps to firm facial muscles with its daily or weekly regimen.

The present invention offers a cutting edge approach to personal skin care. The present invention acts as a real mini face lift with immediate results, able to stabilize skins biological structure and increase its elasticity to give it a luminous tone. One single device eliminates expensive treatments at a doctor's office or at a Spa rendering them useless, and now you can get a facial at the comfort of home. This breakthrough device rejuvenates the skin and helps solve problems of dry and oily skin.

The present invention's effective treatment is based on ultrasound, ion delivery and far infrared rays, the beneficial energy of the ions attack the bacteria of the skin and strengthens the capabilities of cellular resistance to increase skin elasticity, reduces visibility of pores and smoothes the skin, Far Infrared Rays improves the circulation of blood vessels and speeds skin metabolism, the ultrasound's micro massage feature strengthens the elasticity of the skin, rendering skin younger and more luminous.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A skin care apparatus comprising:
an infrared ray generator generating an infrared ray generator;
an ultrasound generator generating ultrasound;
an ion delivery device generating and delivering an ion;
a first output part through which the generated ultrasound and the generated ion are outputted;
a second output part through which the generated infrared ray is outputted; and
a controller controlling the infrared ray generator, the ultrasound generator, and the ion delivery device in accordance with a preprogrammed mode or in accordance with the user's selection, the controller capable of operating concurrently at least two of the infrared ray generator, the ultrasound generator, and the ion delivery device.

2. The skin care apparatus of claim 1, wherein the infrared ray generator generates far-infrared ray with the wavelength of 8-14 μm.

3. The skin care apparatus of claim 1, wherein the ultrasound generator generates about 5 million vibrations per second.

4. The skin care apparatus of claim 1, wherein the ion delivery device generates galvanic ions.

5. The skin care apparatus of claim 1, wherein the controller is capable of operating the ultrasound generator and the infrared ray generator concurrently and operating the ion delivery device and the ultrasound generator concurrently.

6. The skin care apparatus of claim 1, wherein the preprogrammed mode comprises a spot care mode and a wrinkle care mode.

7. The skin care apparatus of claim 6, further comprising a wrinkle care mode switch for selecting the wrinkle care mode and a spot care mode switch for selecting the spot care mode.

8. The skin care apparatus of claim 1, further comprising a tri-function indicator to indicate a status of the infrared ray generator, a status of the ultrasound generator, and a status of the ion delivery device.

9. A method of skin care using the skin care apparatus of claim 1, the method comprising:
removing and arranging keratin by operating the ultrasound generator and the infrared ray generator concurrently;
performing at least one of whitening a skin of a user by operating the ion delivery device and the ultrasound generator concurrently and caring wrinkles by vibration and ultrasonic wave generated from the ultrasound generator; and
supplemental-treating the skin by vibration after whitening and by vibration and ultrasonic wave generated by the ultrasound generator after caring the wrinkles.
